(19) United States
(12) Patent Application Publication
(10) Pub. No.: US 2025/0235537 A1
(43) Pub. Date: Jul. 24, 2025

(54) UTILIZATION OF NANOPARTICLES IN TARGETED THERAPY

(71) Applicant: Alcon Inc., Fribourg (CH)

(72) Inventor: Paul R. HALLEN, Colleyville, TX (US)

(21) Appl. No.: 19/033,776

(22) Filed: Jan. 22, 2025

Related U.S. Application Data
(60) Provisional application No. 63/624,020, filed on Jan. 23, 2024.

(51) Int. Cl. A61K 41/00; A61K 9/00; A61K 9/14

(52) U.S. Cl. CPC A61K 41/0052; A61K 9/0019; A61K 9/0048; A61K 9/14; A61K 41/0057

(57) ABSTRACT

Methods for targeted therapy are disclosed. In certain embodiments, a method includes injecting nanoparticles into a target site in or adjacent to an eye, irradiating the target site with light from a light source, and activating the nanoparticles with the light.

### CROSS-REFERENCE TO RELATED APPLICATIONS

This application claims the benefit of U.S. Provisional Application Ser. No. 63/624,020(filed on Jan. 23, 2024), the content of which is incorporated herein by reference in its entirety.

### INTRODUCTION

This section provides background information to facilitate a better understanding of the various aspects of the disclosure. It should be understood that the statements in this section of this document are to be read in this light, and not as admissions of prior art.

Ophthalmic/ocular tumors are benign or cancerous growths that develop in portions of the eye (e.g., the vitreous, retina, uvea, iris, etc.). These growths may originate within the eye or spread to the eye from other locations in the body. Types of ophthalmic tumors may include uveal melanoma, retinoblastoma, lymphoma, or ocular surface squamous neoplasia. Tumors in the eye may be treated with chemotherapy, radiation, and/or surgical excision. However, current treatment modalities may be imprecise leading to bleeding, pain, and/or infections at the treatment site.

### SUMMARY

This summary is provided to introduce a selection of concepts that are further described below in the Detailed Description. This summary is not intended to identify key or essential features of the claimed subject matter, nor is it to be used as an aid in limiting the scope of the claimed subject matter.

In one or more embodiments, a method for targeted therapy is disclosed. The method includes injecting nanoparticles into a target site in or adjacent to an eye, irradiating the target site with light from a light source, and activating the nanoparticles with the light.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates an example eye.

FIG. 2 illustrates an example method for targeted therapy for treatment of a tumor in the eye of FIG. 1, according to certain embodiments.

FIG. 3 illustrates an increase in porosity of a tumor after exposing the tumor to heat, according to certain embodiments.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Ophthalmic/ocular tumors (eye tumors) encompass a range of benign and malignant growths that can occur in various parts of the eye, such as the vitreous, the retina, a sub-retinal area, the uvea, the ciliary body, the choroid, the eyelid, the iris, or optic nerves of the eye. These tumors may either originate within the eye or spread to the eye from other parts of the body. The development of eye tumors may significantly impact vision and may present serious health challenges. Types of eye tumors may include, without limitation, uveal melanoma, retinoblastoma, lymphoma, or ocular surface squamous neoplasia. Therapy options for eye tumors may include, for example, surgery, radiation therapy, chemotherapy, immunotherapy, among other options. However, traditional therapies for treating ocular tumors can be imprecise. The areas of the eye where ocular tumors general grow may cover a small area that is densely populated with healthy and important tissue. Imprecise treatment therapies may inadvertently damage healthy tissue causing pain, bleeding, swelling, infections, and the like. As a result, more precise therapeutic treatments are greatly needed. This may be particularly relevant for the eye where tissue that is damaged (such as the optic nerve) may not recover, and irreparable damage to ocular tissue can have a much more significant impact on quality of life relative to other areas of the body.

Light-based therapies, such as laser therapy, leverage interactions between light and tissue cells to achieve therapeutic effects, offering a promising avenue for minimally invasive and targeted treatments for ocular tumors. Laser therapy offers a highly localized treatment area leading to more precise therapy thus allowing for the treatment of tumors densely surrounded by healthy tissue.

While laser therapy provides an improvement over conventional therapies, the targeted areas of treatment may suffer from a lack of absorbed heat and/or energy thereby minimizing efficacy. For example, during laser treatment of ocular tumors, more laser power (energy delivered by a laser beam per unit of time and per unit of area) is often needed. Standard approaches to laser therapy in the eye may fail to meet the required laser power.

As such, aspects of the disclosure pertain to utilization of nanoparticles to increase heat and/or energy absorption during therapies, such as, photodynamic therapy (PDT), photothermal therapy (PTT), laser and/or light therapy, ablation therapy, or the like.

Additionally and/or alternatively, as nanoparticles may increase heat and/or energy absorption, this may additionally increase porosity of a tumor. An increase in porosity of the tumor may increase the ability of the tumor to absorb medicaments, thus enhancing targeted drug therapy via increased absorption of the medicament. As such, aspects of the disclosure relate to targeted drug therapy utilizing light sources.

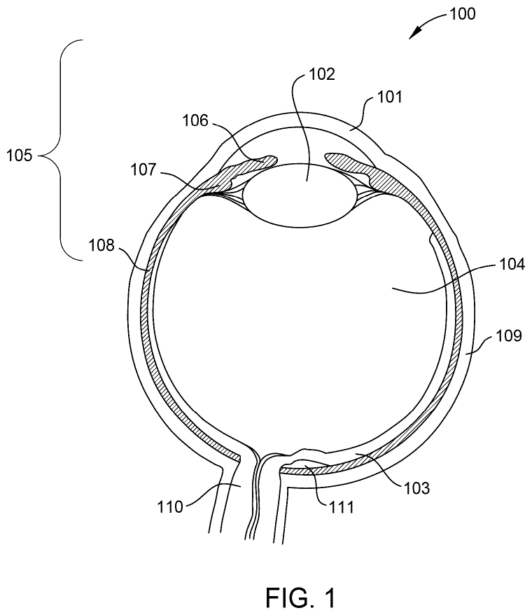

FIG. 1 illustrates an example eye 100. As shown in FIG. 1, the eye 100 includes a cornea 101 and lens 102 that focus light onto light-sensitive cells of the retina 103. The space between the lens 102 and the retina 103 is filled with a transparent gel-like fluid known as the vitreous 104. A sub-retinal area 111 is located between non-light-sensitive cells (e.g., retinal pigment epithelium) and photoreceptors in the retina 103. The uvea 105 is made up of iris 106, ciliary body 107 and choroid 108, and is located beneath sclera 109. The back of the eye 100 includes optic nerve 110 that relays messages from the eye 100 to the brain.

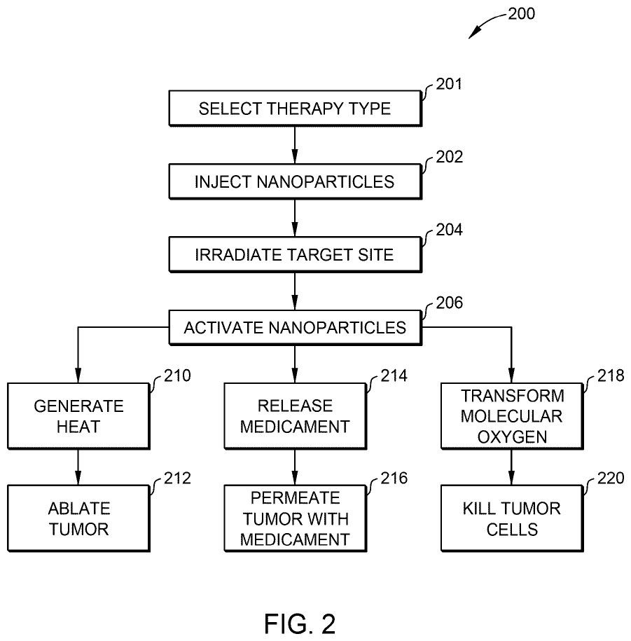

FIG. 2 illustrates an example method 200 for targeted therapy for treatment of an ophthalmic/ocular tumor in the eye 100. While the method 200 is described with respect to an ocular tumor (e.g., ocular cancer), other types of eye conditions and/or diseases are readily envisioned. In certain embodiments, the method 200 may be performed in an operating room. In some other embodiments, the method 200 may be performed in an office. At step 201, an ophthalmologist, ocular oncologist, and/or other doctor and/or surgeon may select a type of targeted therapy to be performed on a patient. For example, in some embodiments, the targeted therapy may include, for example, photodynamic therapy (PDT) and photothermal therapy (PTT), laser and/or light therapy, ablation therapy, drug therapy (e.g., targeted drug therapy), and combinations of the same and like.

In certain embodiments, the targeted therapy may be chosen based on, for example, tumor type, whether the tumor is benign or malignant, types of medicaments desired, location of tumor (e.g., iris 106, uvea 105, etc.), depth of the tumor in the eye 100, and combinations of the same and like. In some embodiments, the type of targeted therapy selected at step 201 may correspond to a type of nanoparticle and/or light source as discussed herein.

At step 202, the surgeon injects nanoparticles into a target site in or adjacent to the eye 100. In some embodiments, the target site may include the tumor and/or areas around the tumor or another portion of the eye 100. In some embodiments, the target site may be on the inside and/or on the surface of the tumor. In certain embodiments, the target site may include the vitreous 104, the retina 103, a sub-retinal area 111, the uvea 105, the ciliary body 107, the choroid 108, and/or the iris 106 of the eye 100. In some embodiments, the nanoparticles may be injected via targeted injection (e.g., directly in the tumor). In certain embodiments, the nanoparticles are injected into the target site via a pneumatic injection device or a manual injection device.

In certain embodiments, the nanoparticles may have an absorption spectrum corresponding to a type of light wavelength and/or source. As nanoparticles may have stronger or weaker absorption at certain wavelengths of light, various properties associated with the nanoparticles may be configured and/or selected to optimize treatment of the eye 100. For example, in certain embodiments, the nanoparticles may have various shapes that may include, without limitation, nanospheres, nanorods, nanotubes, nanoshells, nanoflakes, nanoplates, thermal nano-architectures, other nanostructures with porous walls and a hollow interior, and combinations of the same and like. In certain embodiments, the nanoparticles may include nano-architectures having a form that may include, for example, nanocages.

Furthermore, in certain embodiments, the nanoparticles may have various sizes (e.g., diameter and/or surface area) that may vary, for example, from ˜1-150 nm, or from ˜1-500 nm. As an example, in certain embodiments, the nanoparticles may include nanospheres having a diameter of ˜1-100 nm. In some embodiments, the nanoparticles may include nanorods having a length of ˜10-120 nm. In some embodiments, the nanoparticles may include nanotubes having a length of ˜10-120 nm. In certain embodiments, the nanoparticles may include nanoshells having a diameter of ˜50-150 nm. In some embodiments, the shape and/or size of the nanoparticles may influence distribution of the nanoparticles, and thus heat energy distribution, in the eye 100.

Additionally and/or alternatively, in some embodiments, different materials of the nanoparticles may enhance the conversion of light energy to heat energy by increasing light absorption at a specific wavelength. As such, in certain embodiments, the nanoparticles may include, without limitation, gold nanoparticles, silver nanoparticles, platinum nanoparticles, plasmonic nanoparticles, or combinations of the same and like. In certain embodiments, the nanoparticles may be any biocompatible and/or stable material exhibiting light-to-heat conversion properties.

In certain embodiments, when the selected targeted therapy includes drug therapy, the nanoparticles may include a medicament. In some embodiments, the medicament may be disposed in a coating on the nanoparticles. In certain embodiments, the medicament is attached to the nanoparticles via functional groups on the nanoparticles (e.g., functionalized nanoparticles). In certain embodiments, the medicament is encapsulated within the nanoparticles (e.g., nanospheres, nanocages, or thermal nano-architectures).

In some embodiments, the medicaments target proteins that control tumor cell growth, division, and/or proliferation. In some embodiments, targeted therapy may be desired in areas of the eye 100 where healthy tissue may be damaged during PTT, PDT, and/or laser and/or light therapy. Medicaments for targeted treatment therapy may include, for example, small-molecule drugs and/or monoclonal antibodies. In certain embodiments, the targeted therapy may include blocking blood cell growth that may support tumor growth as opposed to blocking the growth of tumor itself. As such, in some embodiments, the medicament may include, for example, an antiangiogenic drug (e.g., angiogenesis inhibitors). In some embodiments, the medicament is bevacizumab.

In certain embodiments, when the selected targeted therapy is PDT, the nanoparticles may include, for example, a photosensitizer that may be activated by a specific wavelength of light. In some embodiments, the photosensitizer may be separately injected at the target site before and/or after injection of the nanoparticles. Photosensitizers may produce reactive oxygen species and/or free radicals when irradiated with light having a wavelength operable to excite the photosensitizers. Upon the excitation of the photosensitizer, the photosensitizer may undergo intersystem crossing (a process that arises from spin-orbit coupling between electron states belonging to different electron spin multiplicities) to produce singlet oxygen, a reactive oxygen species that is cytotoxic. Oxidative stress may occur within tumor cells as a result of an imbalance between production and/or accumulation of reactive oxygen species and the ability of the body to remove the reactive oxygen species. Such oxidative stress may damage and/or kill cells.

At step 204, the target site is irradiated with light from a light source corresponding to the absorption wavelength of the nanoparticles. In various embodiments, the light source may have a light wavelength operable to reach a target penetration depth needed to reach the tumor. For example, targeted therapies may utilize light in the visible light spectrum when a shallower penetration depth is desired, and thus the light source may include light-emitting diodes (LEDs). In certain embodiments, the light source may be a near-infrared (NIR) light source when a deeper level of penetration is desired. In some embodiments, the light source may have a light wavelength that corresponds to a desired heat conversion temperature at the target site. In certain embodiments, the light source may include, for example, an infrared (IR) light source, an ultraviolet (UV) light source, a laser (e.g., 20 megawatts (mW)), a super-luminescent diode, a diachronic light source, a fluorescent light source, or combinations of the same and like.

In certain embodiments, at or before step 204, the method 200 may include directing the light via an optical coherence tomography (OCT) guide to the target site. OCT techniques offer non-invasive methods to produce cross-sectional images of portions of the eye 100 (e.g., retina 103). In some embodiments, OCT may provide micrometer-level depth resolution imaging in real time. As such, OCT may be utilized in combination with the method 200 to provide high precision (e.g., micrometer level) targeting of the light. Additionally and/or alternatively, in some embodiments, OCT techniques may also be implemented at step 202 to facilitate the injection and/or placement of the nanoparticles within at the target site.

At step 206, the nanoparticles may be activated with the light. In some embodiments, the activating the nanoparticles increases light absorption at the target site. Based on the type of targeted therapy desired, various processes may ensue at the step 206. Steps 210-212, 214-216, and 218-220 are examples of different processes that may be performed as all or part of the step 206, each of which is described below. The aforementioned processes may, in some embodiments, be performed individually or, in other embodiments, as a group of processes performed concurrently.

With reference to steps 210-212, in certain embodiments, at step 210, the activating may include generating heat at the target site through light absorption of the nanoparticles responsive to the irradiation at step 204. In some embodiments, irradiation may be performed for a duration of time such that permeability increases at the target site to a target porosity. In certain embodiments, irradiation may be performed for a duration of time such that all target tissue of the tumor in the target site has been ablated. In some embodiments, the activating may include heating the nanoparticles with a laser having a power ranging from 10 mW to 99 mW (e.g., in order to increase porosity at the target site). In some embodiments, the activating may include heating the nanoparticles with a laser having a power ranging from 100 mW to 3 W (e.g., in order to ablate tumor cells). In certain embodiments, heat is generated at the target site through light absorption and the conversion of light energy to heat energy by the nanoparticles. In some embodiments, at step 212, the heat generated by the activation of the nanoparticle may ablate the tumor.

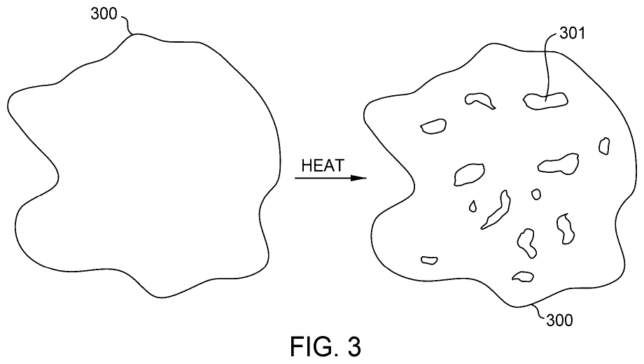

With reference now to steps 214-216, in certain embodiments, activating the nanoparticles may cause a porosity of the tumor to increase responsive to the irradiation at step 204. In some embodiments, the porosity is increased in the tumor as heat is generated by conversion of light energy to heat energy. The increase in porosity of the tumor may result in higher absorption of medicaments. As such, in some embodiments, at step 214, the activating may include releasing the medicament at the target site. In certain embodiments, the medicament may adhere to and/or migrate to the tumor after release. At step 216, the medicament permeates through the target site. FIG. 3 illustrates an example tumor 300 before and after heating. In certain embodiments, the tumor 300 may have a low porosity, thus hindering the ability of the tumor 300 to absorb medicaments when targeted therapy is performed. As illustrated in FIG. 3, when the tumor 300 is subjected to heat, pores 301 develop within the tumor 300. The pores 301 may, for example, increase absorption properties of the tumor 300, thus allowing for higher absorption of medicaments within the tumor 300.

With reference now to steps 218-220 of FIG. 2, in certain embodiments, nanoparticles may increase the absorption of light energy at the target site, responsive to the irradiation at step 204, which may improve the ability of the photosensitizer to produce reactive oxygen species and/or free radicals. As such, in certain embodiments, at step 218, the method 200 may include transforming molecular oxygen into reactive oxygen species. At step 220, the reactive oxygen species may kill tumor cells via oxidative stress caused by, for example, an overabundance of reactive oxygen species at the target site. In some embodiments, the photosensitizer may be separately injected at the target site before and/or after injection of the nanoparticles.

While step 206 is described as single group of processes, it should be understood that each group of processes branching from step 206 may be performed in sequence or in parallel. For example, in certain embodiments, the method 200 may simultaneously generate heat and release a medicament, thus enabling medicament delivery while also ablating portions of the tumor.

In certain embodiments, targeted therapies such as those outlined above can provide for a highly localized therapy treatment which increases the precision of treatment therefore. This precision allows for treatment even when the tumor is densely surrounded by healthy tissue. Furthermore, the targeted therapies outlined above may provide an increase in heat energy at the target site when compared to treatments without nanoparticles. Increased energy at the target site allows for increased efficacy of the treatment. Additionally and/or alternatively, the increased energy may also increase porosity of tumors in the target area to allow higher concentration of medicament delivery to the tumor.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those of ordinary skill in the art may better understand the aspects of the disclosure. Those of ordinary skill in the art should appreciate that they may readily use the disclosure as a basis for designing or modifying other processes and structures for carrying out the same purposes and/or achieving the same advantages of the embodiments introduced herein. Those of ordinary skill in the art should also realize that such equivalent constructions do not depart from the spirit and scope of the disclosure, and that they may make various changes, substitutions, and alterations herein without departing from the spirit and scope of the disclosure. The scope of the disclosure should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements, and/or states are in any way required for one or more embodiments.

While the above detailed description has shown, described, and pointed out novel features as applied to various embodiments, it will be understood that various omissions, substitutions, and changes in the form and details of the embodiments illustrated can be made without departing from the spirit of the disclosure. As will be recognized, the various embodiments described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. A method for targeted therapy, the method comprising:
injecting nanoparticles into a target site in or adjacent to an eye;
irradiating the target site with light from a light source; and
activating the nanoparticles with the light.

2. The method of claim 1, wherein the nanoparticles comprise at least one of nanospheres, nanorods, nanoshells, nanotubes, nanoflakes, nanoplates, or thermal nano-architectures.

3. The method of claim 1, wherein the nanoparticles comprise at least one of gold nanoparticles, silver nanoparticles, platinum nanoparticles, or plasmonic nanoparticles.

4. The method of claim 1, wherein the target site comprises a tumor.

5. The method of claim 4, wherein the activating the nanoparticles comprises generating heat in the tumor responsive to the irradiating.

6. The method of claim 4, wherein activating the nanoparticles causes a porosity of the tumor to increase.

7. The method of claim 1, wherein the nanoparticles comprise a medicament.

8. The method of claim 7, wherein the medicament is disposed in a coating on the nanoparticles.

9. The method of claim 7, wherein the medicament is encapsulated within the nanoparticles.

10. The method of claim 7, wherein activating the nanoparticles comprises releasing the medicament at the target site.

11. The method of claim 7, wherein the activating the nanoparticles causes the medicament to release and permeate the target site.

12. The method of claim 7, wherein the medicament comprises an antiangiogenic drug.

13. The method of claim 1, wherein the injecting the nanoparticles comprises targeted injection.

14. The method of claim 1, wherein the nanoparticles are injected into the target site via at least one of a pneumatic injection device or a manual injection device.

15. The method of claim 1, comprising directing the light via an optical coherence tomography (OCT) guide to the target site.

16. The method of claim 1, wherein the target site comprises at least one of a vitreous, a retina, a sub-retinal area, a uvea, a ciliary body, a choroid, or an iris of the eye.

17. The method of claim 1, wherein the light source comprises at least one of an infrared (IR) light source, a near-IR light source, an ultraviolet light source, a laser, a light-emitting diode, a super-luminescent diode, a diachronic light source, or a fluorescent light source.

18. The method of claim 1, wherein the targeted therapy comprises at least one of photodynamic therapy, photothermal therapy, light therapy, ablation therapy, or medicament delivery therapy.

19. The method of claim 1, wherein the activating the nanoparticles causes heat to generate at the target site through light absorption of the nanoparticles and ablate tumor cells within the target site via the generated heat.

20. The method of claim 1, wherein the activating the nanoparticles causes molecular oxygen to transform into reactive oxygen species to kill tumor cells via oxidative stress.
